(19) United States
(12) Patent Application Publication
(10) Pub. No.: US 2003/0233093 A1
(43) Pub. Date: Dec. 18, 2003

(54) Osteogenic distraction device

(76) Inventors: Randall Moles, Racine, WI (US); David E. Paquette, Charlotte, NC (US)

Correspondence Address: JANSSON, SHUPE & MUNGER, LTD, 245 MAIN STREET, RACINE, WI, 53403, US

(21) Appl. No.: 10/170,602

(22) Filed: Jun. 13, 2002

(51) Int. Cl. A61B 17/56

(52) U.S. Cl. 606/60; 606/71; 606/57; 606/105

(57) ABSTRACT

The invention involves an osteogenic mandibular distraction device. The device is particularly suited for bucchal installation. The device comprises a thoracic member having a fore end and an aft end; a tail member slidably engaged to the thoracic member at the aft end thereof, thereby defining a longitudinal axis between the thoracic member and the tail member, and having a tail member fastening feature for attachment with respect to a first boney structure; an axial extender rotatably attached with respect to the thoracic member, extending therefrom in an aft direction, and engaged with respect to the tail member, whereby a distance between the thoracic member and the tail member may be variably established; a head member; and a rotator attached with respect to the fore end of the thoracic member allowing specific and fixable rotational positioning transverse to the longitudinal axis of the head member with respect to the thoracic member. The head member has a rear portion pivotably attached with respect to the fore end of the thoracic member, and a forward portion with a forward fastening feature for attachment with respect to a second boney structure.

### FIELD OF THE INVENTION

This invention relates to orthodontic devices, particularly those involving osteogenic distraction.

### BACKGROUND OF THE INVENTION

Malocclusion is the misalignment of the maxillary and mandibular teeth when the jaw is closed. Severe malocclusion requires orthodontic intervention for proper dentofacial biomechanics. Similarly, it is necessary for orthodontic intervention for congenital micrognathia or mandibular defects due to trauma or disease. If left untreated, malocclusion and other mandibular defects can lead to headaches, pain, disfigurement, premature wearing of the teeth, and eventual tooth loss.

In applications in which the mandible is short relative to the upper jaw (recessive malocclusion), the mandible needs to be distended.

The process of extending the bone is known alternatively as osteogenesis, osteosynthesis, or osteodistraction. In general terms, the living bone to be lengthened is cut perpendicularly to the direction of distraction. The bone is then separated and braced. After new bone has filled the gap created by the distraction and ossified, the brace can be removed.

Under present procedure for mandibular distraction, an adjustable mechanism is installed to the lateral aspect of at least one ramus under the masseter muscles and also attached by surgical pins to the body of the mandible. The mandible is then transected between the points of installation and attachment. Incremental adjustments by application of the device distends the mandible body anteriorly relative to the ramus over time to maintain directional placement as the connective tissue is lengthened to allow for osteogenesis along the line of transection.

There are a number of appliances available to an orthodontist for correcting malocclusion. One type of appliance uses a small screw jack attached to selected teeth. The devices of Guerrero et al. (U.S. Pat. No. 5,885,290) and Gittleman (U.S. Pat. No. 5,885,283) constitute an attempt at providing a distraction device for distention of the mandible. These devices allow for distention along one line of travel.

While the majority of the mandibular distraction motion is in an anterior direction along one axis, malformations and traumatic reconstructions often require movement along other than a single linear direction.

The device of Schendel (U.S. Pat. No. 5,700,263) allows for bidirectionality in adjustment. Such bidirectionality, however, must be predetermined prior to installation of the apparatus. Moreover, the Schendel device does not allow for different rates of distraction between the various dimensional adjustments.

An osteogenic distraction device facilitating easy adjustment in each of two dimensions, adjustment in either dimension independent of adjustment in the other dimension, would be an important improvement in the art.

### OBJECTS OF THE INVENTION

It is an object of the invention to provide an osteogenic distraction device that overcomes some of the shortcomings of the prior art.

It is another object of this invention to provide an osteogenic distraction device which is easy to install to a mandible.

It is still another object of this invention to provide an osteogenic distraction device which is easily adjustable.

It is yet another object of this invention to provide an osteogenic distraction device which is easily adjustable by a patient into whom a device has been implanted.

It is still yet another object of this invention to provide an osteogenic distraction device which is easily customizable by a surgeon before placement of a device into a patient.

It is a further object of this invention to provide an osteogenic distraction device which once implanted does not need invasive surgery for removal.

It is a still further object of this invention to provide an osteogenic distraction device which minimizes the chance of infection once implanted within the body.

Another object of this invention is to present an osteogenic distraction device which allows for easy adjustment within either of two dimensions independent of any adjustment in the other dimension.

Yet another object of this invention is to present an osteogenic distraction device which minimizes the number of fixation devices necessary to adequately anchor the distraction device to the patient.

Still another object of this invention is to provide an apparatus and method for minute, incremental distraction adjustments to maximize bone growth and establish long-term bone health.

Even yet another object of the invention is to maintain a union of two sections of bone as the connective tissue is lengthened and new bone is created within the transected gap.

How these and other objects are accomplished will become apparent from the following descriptions and from the drawings.

### SUMMARY OF THE INVENTION

The invention involves an osteogenic mandibular distraction device. The device is particularly suited for bucchal installation. The device comprises a thoracic member having a fore end and an aft end; a tail member slidably engaged to the thoracic member at the aft end thereof, thereby defining a longitudinal axis between the thoracic member and the tail member, and having a tail member fastening feature for attachment with respect to a first boney structure; an axial extender rotatably attached with respect to the thoracic member, extending therefrom in an aft direction, and engaged with respect to the tail member, whereby a distance between the thoracic member and the tail member may be variably established; a head member; and a rotator attached with respect to the fore end of the thoracic member allowing specific and fixable rotational positioning transverse to the longitudinal axis of the head member with respect to the thoracic member. The head member has a rear portion pivotably attached with respect to the fore end of the thoracic member, and a forward portion with a forward fastening feature for attachment with respect to a second boney structure. In this way, variable bidirectional adjustment may be obtained between the tail member fastening feature and the forward fastening feature. By the term “bidirectional adjustment”, it is meant that adjustment may be made independently in either linear direction, or cooperatively to obtain a rotational movement within a plane.

In one preferred embodiment, at least one of the fastening features is a plate extending out from the device, into which at least one hole may be created. It is more preferable for the at least one plate to be bioabsorbable. By “bio-absorbable”, it is meant that the material of which the plate is made, will, over time, be broken down within the body (human or otherwise) into which it is placed, and be absorbed or adsorbed, ultimately being expelled by natural organic processes, without the need for surgical removal.

It is also preferable in this embodiment for the at least one plate to be alterable after manufacture but before installation. It is yet more preferable to have at least one pre-placed discontinuity to allow for separation along the discontinuity when purposeful pressure is applied perpendicularly to the plate. This discontinuity could be, by way of illustration only, and not limitation, a perforation or scoring.

In another preferred embodiment, the at least one plate is a mesh which inherently provides a plurality of screw-receiving ports.

In another preferred embodiment the axial extender is threaded. It is more preferable when the axial extender is a jack screw.

In yet another preferred embodiment, the rotator is threaded. In a different preferred embodiment, the rotator is geared.

It is still yet another embodiment of this osteogenic mandibular distraction device to further comprise: (a) at least one guide having a cross-sectional shape extending from the aft end of the thoracic member; and (b) at least one guide receptor on the tail member with a receptor space of a shape complementary to the guide cross-sectional shape. In this way, the tail member will slide along the guide as the relative distance between the thoracic member and the tail member is varied.

It is another aspect of this invention to provide a method of osteogenic mandibular distraction. This method comprises the steps of:

(a) obtaining a osteogenic mandibular distraction device having: (i) a thoracic member having a fore end and an aft end; (ii) a tail member slidably engaged to the thoracic member at the aft end thereof, thereby defining a longitudinal axis between the thoracic member and the tail member, and having a tail member fastening feature for attachment with respect to a first boney structure; (iii) an axial extender rotatably attached with respect to the thoracic member, extending therefrom in an aft direction, and engaged with respect to the tail member, whereby a distance between the thoracic member and the tail member may be variably established; (iv) a head member; and (v) a rotator attached with respect to the fore end of the thoracic member allowing specific and fixable rotational positioning transverse to the longitudinal axis of the head member with respect to the thoracic member;

(b) severing the bone of the mandible in a transverse band thereby separating a labial portion of the mandible from a cranial portion of the mandible;

(c) fixing the forward fastening feature with respect to the bone on one side of the separation in the mandible;

(d) adjusting the rotator to a fixed, preferred rotational position between the head member and the thoracic member;

(e) fixing the tail member fastening feature with respect to the bone on the opposite side of the separation in the mandible; and

(f) over time, incrementally varying the axial distance between the thoracic member and the tail member; thereby producing mandibular distraction. The head member of this osteogenic mandibular distraction device has a rear portion pivotably attached with respect to the fore end of the thoracic member, and a forward portion with a forward fastening feature for attachment with respect to a second boney structure.

It is preferable for the fixation of the fastening features to be accomplished through screws. It is more preferable for the screws to be bioabsorbable.

Another aspect of this invention is an osteogenic distraction device for oral installation. The device comprises: (a) a thoracic member having a fore end and an aft end; (b) a tail member slidably engaged to the thoracic member at the aft end thereof, thereby defining a longitudinal axis between the thoracic member and the tail member, and having a tail member fastening feature for attachment with respect to a first boney structure; (c) an axial extender rotatably attached with respect to the thoracic member, extending therefrom in an aft direction, and engaged with respect to the tail member, whereby a distance between the thoracic member and the tail member may be variably established; (d) a head member having a rear portion pivotably attached with respect to the fore end of the thoracic member, and a forward portion with a forward fastening feature for attachment with respect to a second boney structure; and (e) a rotator attached with respect to the fore end of the thoracic member allowing specific and fixable rotational positioning transverse to the longitudinal axis of the head member with respect to the thoracic member. In this way, variable bidirectional adjustment may be obtained between the tail member fastening feature and the forward fastening feature.

### BRIEF DESCRIPTION OF THE DRAWINGS

The drawings illustrate preferred embodiments which include the above-noted characteristics and features of the invention. The invention will be readily understood from the descriptions and drawings. In the drawings:

FIG. 1 is a top view of the device according to the invention with a starboard tab.

FIG. 2 is a side view of the side of the device of FIG. 1.

FIG. 3 is a end view of the anterior of the device of FIG. 1.

FIG. 4 is a end view of the posterior of the device of FIG. 1.

FIG. 5 is a top view of an alternative embodiment of a device according to the invention.

FIG. 6 is a partial cut-away top view of the device of FIG. 5.

FIG. 7 is a side view of a partially disassembled device of FIG. 5.

FIG. 8 is a cut-away top view of the device of the device of FIG. 5 with the jack screw displaced producing rotation.

FIG. 9 is a top view of the device of FIG. 1 with alternative embodiments of uniting tabs having supplemental fixation plates.

FIG. 10 is a side view of the device of FIG. 9.

FIG. 11 is a top view of the device of FIG. 1 installed in a mandible.

### DETAILED DESCRIPTION OF PREFERRED EMBODIMENTS

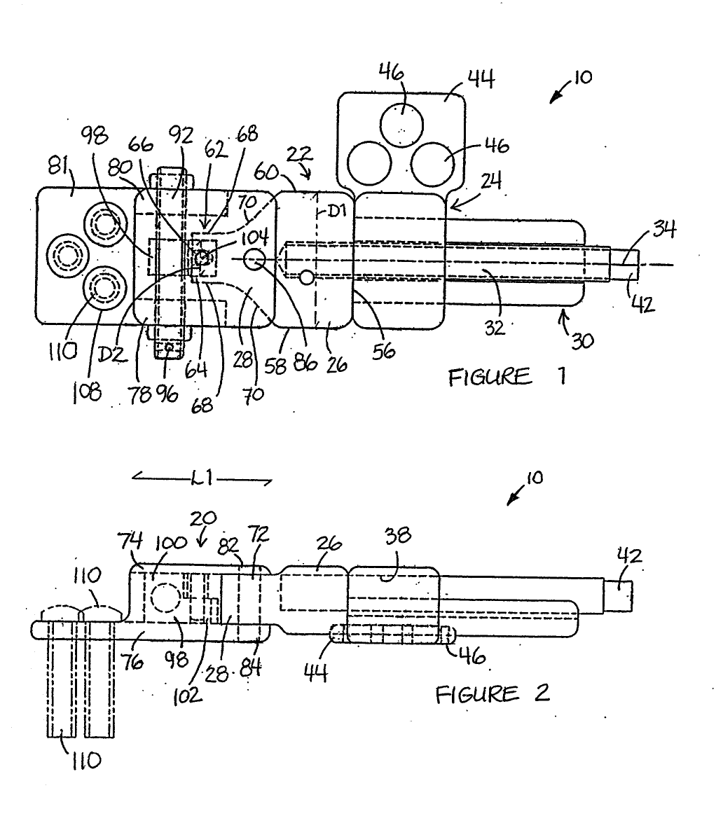

FIG. 1 shows the device 10. Device 10 has a head section 20, a thoracic section 22, and an abdominal section 24. By the terms “head”, “thoracic”, and “abdominal”, applicants are not referring to anatomical parts of the human anatomy, but rather three distinct and aligned members of device 10. For directional convention in describing the device herein, head section 20 is deemed anterior and abdominal section 24 is deemed posterior along a longitudinal dimension.

Head section 20, thoracic section 22, and abdominal section 24 are rigid and must be strong enough to provide the jaw adequate support when the device is installed spanning the transection (as described below) of the mandible while the bone is generating. Unless otherwise discussed, metal and in particular, surgical-grade stainless steel, is preferred for the composition of the components of device 10.

As shown in FIG. 1, when viewed from above, thoracic section 22 has a roughly rectangular posterior thoracic portion 26 and a tapered-neck-shaped anterior thoracic portion 28 (shown in phantom) integral with posterior thoracic portion 26.

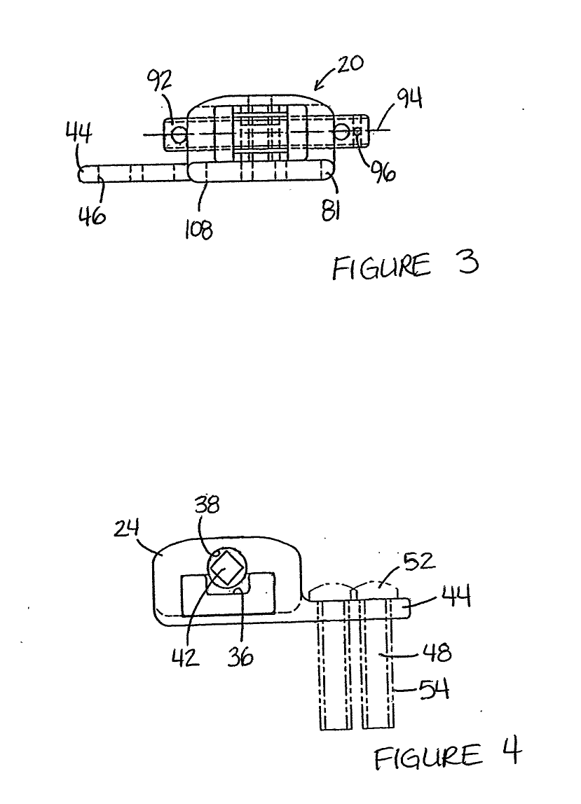

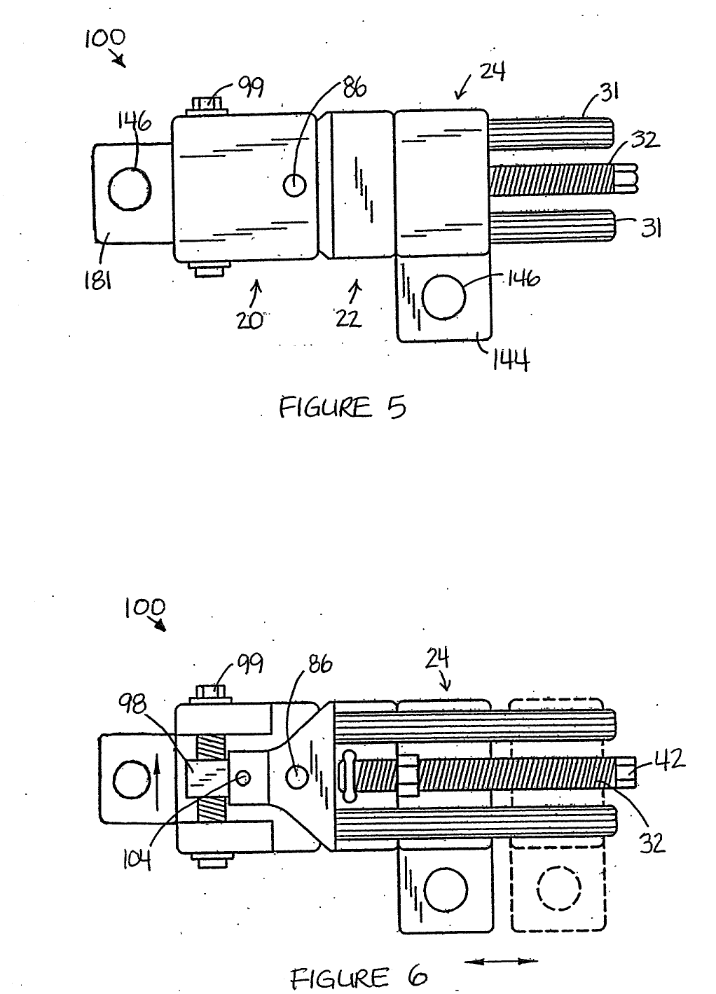

Extending posteriorly out from posterior thoracic portion 26 is a longitudinal motion director. As shown in FIGS. 1-4, longitudinal motion director is a glide 30. Glide 30 is most preferably a U-shaped channel. The purpose of a longitudinal motion director is to allow abdominal section 24 to slide along glide 30 with respect to thoracic section 22 but to constrain the sliding movement to one dimension and prohibit rotation of abdominal section 24 with respect to thoracic section 22. It can be easily understood that any mechanism, such as two parallel, longitudinal bars (31 as shown in FIG. 5) extending posteriorly from thoracic section 22 would provide sufficient structure to keep abdominal section 24 traveling in one dimension with respect to thoracic section 22. It has been found that U-shaped channel glide 30 is best suited for sanitary intra-oral application by eliminating pockets into which debris, microorganisms, or tissue may accumulate.

Also extending posteriorly from thoracic section 22 and parallel to glide 30 is a threaded longitudinal jack screw 32 capable of rotation about a longitudinal jack screw axis 34 without travel into thoracic section 22.

Both glide 30 and jackscrew 32 engage abdominal section 24.

Abdominal section 24, as viewed from the top, is roughly rectangular in shape.

As seen in FIG. 4, extending horizontally through abdominal section 24 from the interior side to the posterior side, are two cavities, the glide-receiving cavity defined by glide-receiving cavity wall 36 and the jack-screw-receiving cavity defined by jack-screw-receiving cavity wall 38. Glide-receiving cavity wall 36 is U-shaped and dimensioned to allow abdominal section 24 to slide along glide 30 but with sufficiently tight tolerance to prevent accumulation of debris and/or tissue in the space between glide 30 and glide-receiving cavity wall 36.

Jack-screw cavity wall 38 is threaded to accommodate longitudinal jack screw 32. At the posterior end of jack screw 32 is adjustment head 42. It is the purpose of adjustment head 42 to allow longitudinal adjustment along longitudinal jack screw axis 34 by turning of a wrench. Any means of turning jack screw 32 about its axis 34 to cause movement of abdominal section 24 along glide 30 is sufficient for purposes of the invention. As seen in FIG. 6, when jack screw 32 is rotated, abdominal section 24 may be moved along longitudinal motion director.

As seen in FIG. 1, when viewed from the top, extending out from abdominal section 24, perpendicular to longitudinal jack screw axis 34 of jack screw 32 is an abdominal fixation tab 44. As better seen FIG. 4, abdominal fixation tab 44 is integral with abdominal section 24. As shown, abdominal fixation tab 44 has three screw-receiving apertures 46. Screw-receiving apertures 46 are designed to receive fixation screws 48 (shown in phantom in FIG. 4). Fixation screws 48 are driven through screw-receiving apertures 46 to allow securing of device 10 to a mandible 50. Fixation screws 48 may be either of surgical-grade stainless steel or alternatively of a biodigestable material for absorption into the body over time. In this latter manner, removing device 10 from the oral cavity is facilitated. Rather than removing each fixation screw 48 from their place of fixation within mandible 50, the screw head 52 may be cut off and device 10 may be removed while screw body 54 remains in the jaw for absorption over time by the human body.

It should be noted that, as shown in FIG. 1, abdominal fixation tabs 44 extend out from the starboard side of abdominal section 24. This version of device 10 may be used on either side of the jaw, although the embodiment shown in FIG. 1 is best suited for buccal installation in the left jaw. Alternatively, for right-side buccal installation, a similarly shaped abdominal fixation tab 44 may extend on the port side of abdominal section 24. In addition, it is easily understood that such abdominal fixation tab 44 may extend axially outwardly from the posterior end of abdominal section 24.

Posterior portion 26 of thoracic section 22 is rectangular with a laterally-running thoracic-section posterior side 56, and has opposed longitudinally-running, parallel, posterior-portion thoracic-section, lateral sides 58, 60 each perpendicular to the thoracic-section posterior side 56. Posterior-portion thoracic-section lateral sides 58, 60 are spaced apart by a lateral dimension D1. Anterior portion 28 of thoracic section 22 engages head section 20 at a post-engaging arrangement 62.

In the preferred embodiment, post-engaging arrangement 62 consists of fork with two tines 64 defining a post-receiving cavity 66. Tines 64 have longitudinally-running tine sides 68 separated by a lateral distance D2 which is approximately one-third of the lateral distance D1. Tying the most posterior point of each tine side 68 to the most anterior point of each posterior-portion thoracic-section lateral side 56 are anterior-portion angled sides 70. Angled sides 70 are positioned approximately 45° from each posterior-portion thoracic-section lateral side 58, 60. By providing an angular distance between post-engaging arrangement 62 and posterior-portion thoracic-section lateral side 56, clearance is provided for rotation of thoracic section 22 with respect to head section 20.

As seen in side view of FIG. 2, anterior portion 28 has a smaller transverse dimension than posterior portion 26. Extending transversely through anterior portion 28 is a pivot cavity 72.

Head section 20 has a roughly rectangular cross-section, comprised of an upper piece 74, lower piece 76, port piece 78, and starboard piece 80 and is substantially hollow. Port piece 78 and starboard piece 80 are dimensioned to provide adequate support for head jack screw 92, but do not extend the full length of head section 20 to provide an opening at the posterior end of head section 20 whereby anterior portion angled sides 70 may travel a distance as thoracic section 22 is pivoted with respect to head section 20, without obstruction by posterior edges of port and starboard pieces 78, 80.

In the preferred embodiment, upper piece 74, lower piece 76, port piece 78, starboard piece 80, and a head tab 81 are integrated to form one, continuous housing with rounded corners and sides. This unibody design of head-section 20 decreases opportunity for tissue-holding pockets and, with the rounded corners and edges, allows for travel along tissues as device 10 is being adjusted in operation within the body.

Extending transversely through a point at the posterior of upper piece 74 and through a point at the posterior of lower piece 76 of head section 20 are upper head pivot hole 82 and lower head pivot hole 84, respectively. Extending through upper head pivot hole 82, pivot cavity 72, and lower head pivot hole 84, is a pivot pin 86. Pivot pin 86 is dimensioned to be flush with the upper surface of upper piece 74 and the lower surface of lower piece 76 to provide comfortable, smooth surfaces for the wearer.

As seen in FIGS. 1 and 2, device 10 (and the similar mechanism shown in an alternate embodiment as device 100 of FIGS. 5-8) includes a head jack screw 92 which is supported by head port piece 78 and head starboard piece 80, and extends laterally through head section 20. Head jack screw 92 is threaded and is capable of being rotated around about head jack screw axis 94 without travel. Rotation of thoracic section 22 in the embodiment shown in FIGS. 1-4, is accomplished by means of a standard key (not shown) removably placed in a key hole 96 to lever torque to head jack screw 92. It is well known in the industry that other methods are equally effective to induce torque. One such alternate method is shown in the embodiment of FIGS. 5-8, wherein rotation of the thoracic section 22 is accomplished by inducing torque through a wrench applied to wrench-receiving head 99.

Attached to head jack screw 92 is a head pivot device 98. Head pivot device 98 has a anterior threaded portion 100 and a posterior post-holding portion 102. Anterior threaded portion 100 engages head jack screw 92. It should be noted that, while in the embodiment of FIGS. 1-4, head pivot device 98 is of a generally “L” shape configuration which complementarily receives post-engaging arrangement 62 which is of a generally inverted “L” shape, in the embodiment of FIGS. 5-8, the anterior portion of thoracic section 22 of FIG. 7 has upper and lower arms 165 extending horizontally out therefrom defining a nub-receiving cavity 167 therebetween. Head pivot device 98 of FIGS. 5-8 includes threaded anterior portion 100, post-holding portion 102 and post 104 that extends through post-receiving cavity 66 of the extreme anterior portion 28 of thoracic section 22. Post-receiving portion 102 has a nub configuration which is complementary with nub-receiving cavity 67. These are but two possible methods of providing rotation of the head section 20 with respect to the thoracic section 22.

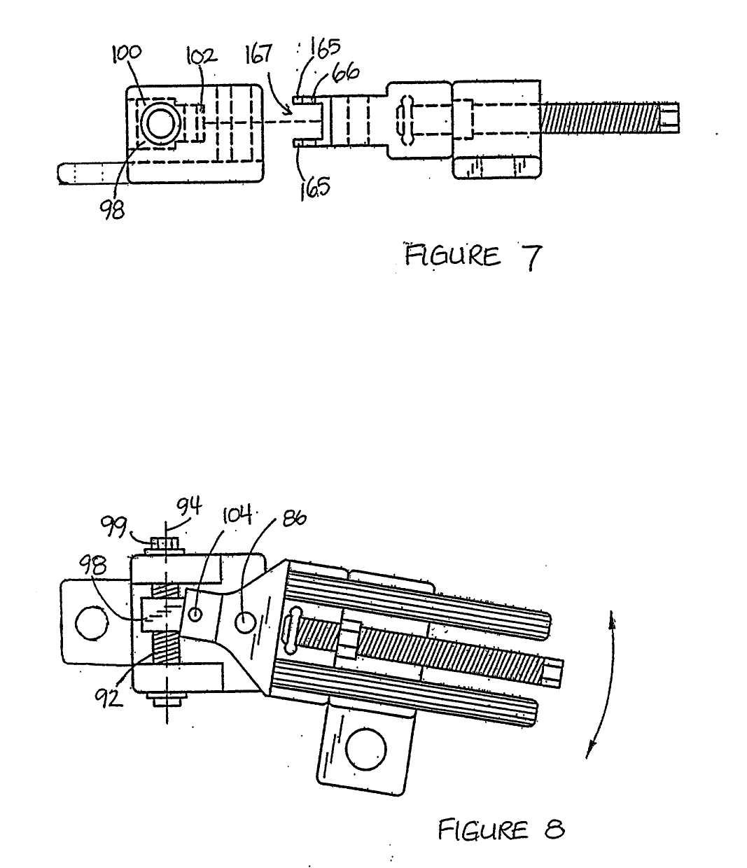

In this manner, as head jack screw 92 is rotated about its axis 94, head pivot device 98 (including post 104) travels along jack screw axis 94, which in turn rotates anterior portion 28 of thoracic section 22 about pivot pin 86 thereby arcuately moving abdominal section 24 with respect to head section 20 (as best seen in FIG. 8).

Extending out anteriorly from head section 20 is head tab 81. Head tab 81 may extend out from head section 20 in any manner which can accommodate fixation to the jaw, such as laterally or radially from head section 20. Head tab 81 has three fixation screw apertures 108 to receive fixation screws 110. As discussed with regard to installation of the abdominal fixation tab 44, head fixation screws 110 may be either stainless steel or bioabsorbable for installation into a jaw.

FIGS. 5-8 illustrate another embodiment of the invention identified as device 100, which includes many common components with device 10, depicts alternative embodiments of the post-engaging arrangement and tail member. For clarity, the components of device 100 which are common with the components of device 10 will be identified by identical numerals. Like device 10, device 100 includes a head section 20, a thoracic section 22, and an abdominal section 24. Device 100 also includes an abdominal fixation tab 144 located on the port side of device 100. As discussed above with regard to fixation tabs 44 of device 10, abdominal fixation tabs 144 may be located on either the port or starboard sides of device 100 to accommodate installation in the mouth. Device 100 is installed within the mouth through the use of a single fixation screw (not shown) extending through screw hole 146 into the jaw. In a similar manner, head tab 181 has one screw hole 146 for a single fixation screw.

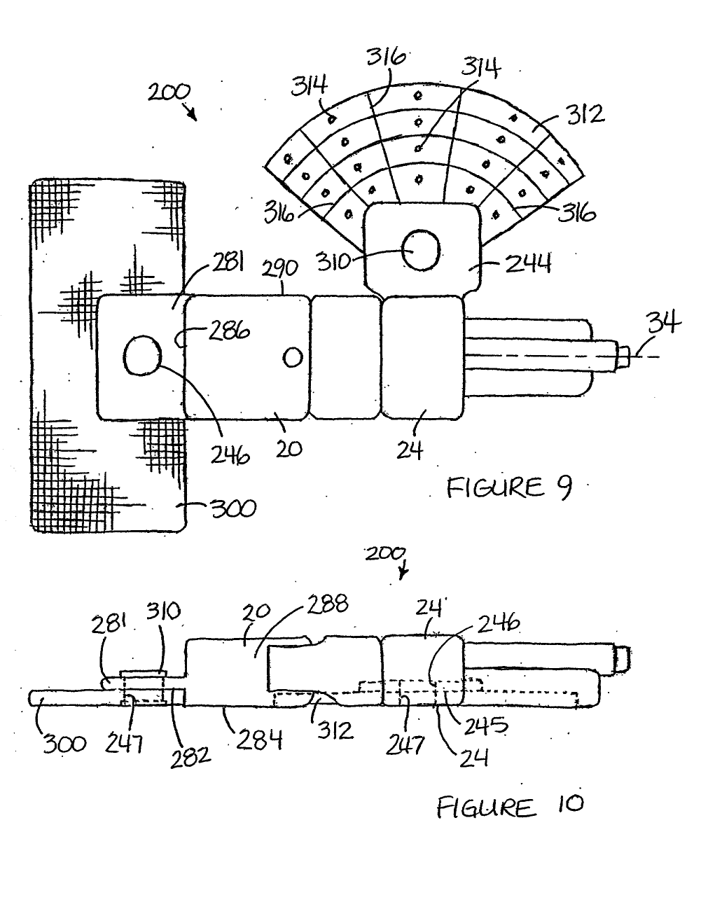

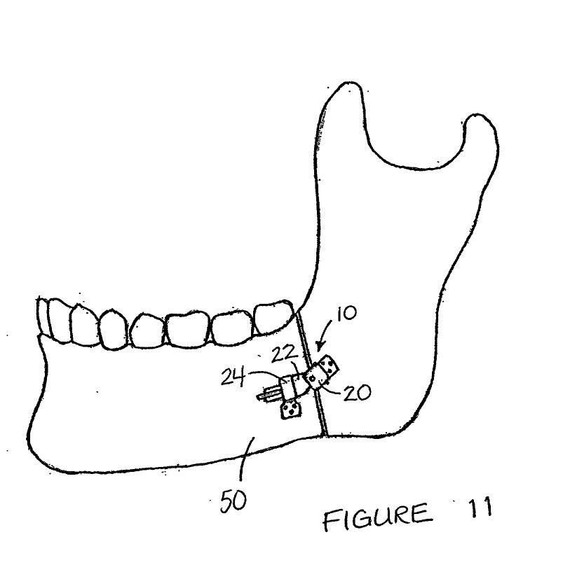

As human mouths come in an infinite array of sizes and shapes with an infinite need for adjustment, it is desirable to provide placement options for the surgeon installing the device. The device 200 depicted in FIGS. 9 and 10 illustrate substantially the same device of FIGS. 1-8 but with alternative types of fixation that is more easily customizable by an installer of the device prior to installation.

Device 200 includes a head uniting tab 281 which extends anteriorly longitudinally from head section 20 and a abdominal uniting tab 244 which extends out from abdominal section 24 (at either port or starboard, as desired), perpendicular to longitudinal jack screw axis 34.

As shown in FIGS. 9-10, head uniting tab 281 has a plate-uniting hole 246. Attached to head uniting tab 281 is an anterior customizing plate 300. Anterior customizing plate 300 has a tab-uniting hole 247. Head uniting tab 281 is secured to customizing plate 300 by a fastener 310 which extends through plate-uniting hole 246 and tab-uniting hole 247. Fastener 310 may be of any suitable type, such as a rivet, and may be made of any suitable material, such as surgical-grade stainless steel or a biodigestable material.

Similarly, abdominal uniting tab 244 has a plate-uniting hole 246 and is likewise secured to a customizing plate 312 by fastener 310. It can be appreciated that, like fixation tabs 44 and 81 of device 10, head uniting tab 281 and abdominal uniting tab 244 may extend out from head section 20 and abdominal section 24, respectively, in any direction which facilitates installation of devices 10, 100 or 200 in the mouth. Futhermore, anterior customizing plate 300 may be secured to head section 20 by any suitable method, such as pinned, screwed or welded to the anterior end wall 286 or a side wall 288, 290 of head section 20. Similarly, customizing plate 312 may be secured to abdominal section 24 in any suitable manner.

In the preferred embodiment shown in FIGS. 9-10, the lower surface 282 of head uniting tab 281 and the lower surface 245 of abdominal uniting tab 244 are located at a distance spaced from the lower surface 284 of head section 20 and the lower surface 248 of abdominal section 24, respectively, approximately equal to the thickness of customizing plates 300, 312 to provide a continuous, flush lower surface along device 200.

Customizing plates 300, 312 may be made of any suitable material with sufficient strength to withstand lateral stresses in treatment operation would be acceptable, most preferrably a surgical-grade plastic or biodigestble material. Customizing plates 300, 312 may be manufactured in any shape or configuration desired, such as a mesh as illustrated by customizing plate 300, or a sheet as illustrated by customizing plate 312. Mesh customizing plate 300 may be trimmed to accommodate or customize the shape thereof to the wearer and further allows the device of the invention to be secured to the wearer by screws at any location desired on mesh customizing plate 300.

Sheet customizing plate 312 may include pre-drilled apertures 314 at predetermined locations thereon to facilitate attachment of the device of the invention to the wearer. Sheet customizing plate 312 may also include scored separation lines 316 which provides easy customization of the shape of sheet customizing plate 312 to accommodate the wearer.

FIGS. 5-8 and 11 also demonstrate a method for osteogenic distraction.

Under anesthetic, an incision is made on the buccal side of the oral cavity allowing the gum to pulled aside providing access to the ramus under the masseter muscle and access to mandible 50. In the preferred method, an initial cut is made vertically along the mandible 50. Prior to complete transection, fixation of the device of the invention within the mouth is accomplished.

Specifically, fixation screws 110 are driven through fixation apertures 108 of head tab 81 into the ramal portion of mandible 50 or into the ramus. While in an undistended position (with thoracic section 22 touching abdominal section 24), abdominal fixation tab 44 (preferably extending downwardly) is affixed to the labial side of the partial transection and affixed to mandible 50 by means of fixation screws 48.

The remainder of the transection is accomplished with care being taken not to disturb the non-boney, filamentary connective tissue portions of mandible 50.

By operation of head jack screw 92 and longitudinal jack screw 32, slight distending adjustment is made thereby separating thoracic section 22 from abdominal section 24 at periodic temporal intervals, at the direction of the surgeon. As additional rotations are provided to longitudinal jack screw 32, the jaw is additionally distended. Slowly widening the gap while maintaining the integrity of the connective-tissue blood supply improves the creation of new bone-forming tissue.

Further, if rotational motion of the jaw is required for proper positioning, in addition to longitudinal distension, torque may be applied to head jack screw 92. As head jack screw 92 rotates in both clockwise and counter-clockwise directions, rotation of the jaw is available upwardly and downwardly about the transection. It will be noted that adjustment may be made by longitudinal jack screw 32 independent of operation of head jack screw 92 thereby allowing for fine adjustments during the course of treatment.

It is preferable to mount device of the invention under the gum, adjacent to the bone, but, it is acceptable to mount the device of the invention superficially to the gum within the oral cavity.

At the conclusion of treatment and after ossification of generated bone in the gulf provided by the transection and distension, the device of the invention will be removed.

All parts can be fashioned from biocompatible stainless steel since this apparatus will remain in place only for up to eight weeks while bone is formed in the lengthening gap. If the device was affixed with metal or other non-bioabsorbable fixation screws 48, fixation screws 48 will be removed from the body thereby releasing the device and allowing it to be removed from the body.

If bioabsorbable screws or pins were used to affix the device to mandible 50, the heads 52 of fixation screws 48 may be cut off thereby allowing for removal of the device with retained placement of the bioabsorbable body 54 of fixation screws 48.

To avoid sharp edges and pain at the site, the remaining core of body 54 of fixation screw 48 may be cut, filed or polished.

Alternatively, if customizable plates such as 300, 312 depicted in FIGS. 9-10 are made of bioabsorbable material along with fixation screws 48, customizable plates 300, 312 may be separated from device 200 at a location anywhere between fixation screws 48 and device 200 to allow for removal of the metallic portion of distension device 200 from the oral cavity. In this latter regard, the body will absorb the bioabsorbable material and allow for natural osteogenic repair of the screw holes.

While the principles of the invention have been shown and described in connection with but a few embodiments, it is to be understood clearly that such embodiments are by way of example and are not limiting.

1. An osteogenic mandibular distraction device for bucchal installation comprising:
a thoracic member having a fore end and an aft end;
a tail member slidably engaged to the thoracic member at the aft end thereof, thereby defining a longitudinal axis between the thoracic member and the tail member, and having a tail member fastening feature for attachment with respect to a first boney structure;
an axial extender rotatably attached with respect to the thoracic member, extending therefrom in an aft direction, and engaged with respect to the tail member, whereby a distance between the thoracic member and the tail member may be variably established;
a head member having:
a rear portion pivotably attached with respect to the fore end of the thoracic member, and
a forward portion having a forward fastening feature for attachment with respect to a second boney structure; and
a rotator attached with respect to the fore end of the thoracic member allowing specific and fixable rotational positioning transverse to the longitudinal axis of the head member with respect to the thoracic member;
whereby variable bidirectional adjustment may be obtained between the tail member fastening feature and the forward fastening feature.

2. The osteogenic mandibular distraction device of claim 1 wherein at least one of the fastening features is a plate extending out from the device having at least one hole.

3. The osteogenic mandibular distraction device of claim 2 wherein the at least one plate is bioabsorbable.

4. The osteogenic mandibular distraction device of claim 2 wherein the at least one plate is alterable after manufacture but before installation.

5. The osteogenic mandibular distraction device of claim 4 wherein the at least one plate has at least one pre-placed discontinuity to allow for separation along the discontinuity when purposeful pressure is applied perpendicularly to the plate.

6. The osteogenic mandibular distraction device of claim 1 wherein at least one of the fastening features is a mesh extending out from the device.

7. The osteogenic mandibular distraction device of claim 1 wherein the axial extender is threaded.

8. The osteogenic mandibular distraction device of claim 7 wherein the axial extender is a jack screw.

9. The osteogenic mandibular distraction device of claim 1 wherein the rotator is threaded.

10. The osteogenic mandibular distraction device of claim 1 wherein the rotator is geared.

11. The osteogenic mandibular distraction device of claim 1 further comprising:
at least one guide having a cross-sectional shape extending from the aft end of the thoracic member, and
at least one guide receptor on the tail member with a receptor space of a shape complementary to the guide cross-sectional shape,
whereby the tail member will slide along the guide as the relative distance between the thoracic member and the tail member is varied.

12. A method of osteogenic mandibular distraction comprising the steps of:
obtaining a osteogenic mandibular distraction device having:
a thoracic member having a fore end and an aft end;
a tail member slidably engaged to the thoracic member at the aft end thereof, thereby defining a longitudinal axis between the thoracic member and the tail member, and having a tail member fastening feature for attachment with respect to a first boney structure;
an axial extender rotatably attached with respect to the thoracic member, extending therefrom in an aft direction, and engaged with respect to the tail member, whereby a distance between the thoracic member and the tail member may be variably established;
a head member having:
a rear portion pivotably attached with respect to the fore end of the thoracic member, and
a forward portion having a forward fastening feature for attachment with respect to a second boney structure; and
a rotator attached with respect to the fore end of the thoracic member allowing specific and fixable rotational positioning transverse to the longitudinal axis of the head member with respect to the thoracic member;
severing the bone of the mandible in a transverse band thereby separating a labial portion of the mandible from a cranial portion of the mandible;
fixing the forward fastening feature with respect to the bone on one side of the separation in the mandible;
adjusting the rotator to a fixed, preferred rotational position between the head member and the thoracic member;
fixing the tail member fastening feature with respect to the bone on the opposite side of the separation in the mandible; and
over time, incrementally varying the axial distance between the thoracic member and the tail member;
thereby producing mandibular distraction.

13. The method of osteogenic mandibular distraction of claim 12 wherein the fixation of the fastening features is accomplished through screws.

14. The method of osteogenic mandibular distraction of claim 13 wherein the screws are bioabsorbable.

15. An osteogenic distraction device for oral installation comprising:
a thoracic member having a fore end and an aft end;
a tail member slidably engaged to the thoracic member at the aft end thereof, thereby defining a longitudinal axis between the thoracic member and the tail member, and having a tail member fastening feature for attachment with respect to a first boney structure;
an axial extender rotatably attached with respect to the thoracic member, extending therefrom in an aft direction, and engaged with respect to the tail member, whereby a distance between the thoracic member and the tail member may be variably established;
a head member having:
a rear portion pivotably attached with respect to the fore end of the thoracic member, and
a forward portion having a forward fastening feature for attachment with respect to a second boney structure; and
a rotator attached with respect to the fore end of the thoracic member allowing specific and fixable rotational positioning transverse to the longitudinal axis of the head member with respect to the thoracic member;
whereby variable bidirectional adjustment may be obtained between the tail member fastening feature and the forward fastening feature.
